(19) United States
(12) Patent Application Publication
(10) Pub. No.: US 2009/0186810 A1
(43) Pub. Date: Jul. 23, 2009

(54) POTASSIUM CHANNEL MODULATORS AND PLATELET PROCOAGULANT ACTIVITY

(75) Inventors: Robert F.A. Zwaal, Meerssen (NL); Pamela B. Conley, Palo Alto, CA (US)

Correspondence Address: FOLEY & LARDNER LLP, 975 PAGE MILL ROAD, PALO ALTO, CA, 94304, US

(73) Assignee: Portola Pharmaceuticals, Inc.

(21) Appl. No.: 12/280,531

(22) PCT Filed: Mar. 26, 2007
(86) PCT No.: PCT/US07/64956

§ 371 Date: Dec. 5, 2008

Related U.S. Application Data
(60) Provisional application No. 60/786,163, filed on Mar. 27, 2006.
(60) Provisional application No. 60/794,106, filed on Apr. 24, 2006.

(51) Int. Cl. A61K 38/12; G01N 33/48; A61K 38/17; A61K 31/519; A61K 31/439; A61K 31/4164

(52) U.S. Cl. 514/11; 436/63; 514/12; 514/262.1; 514/305; 514/396

(57) ABSTRACT

Methods of modulating platelet procoagulant response are taught herein. Inhibition of platelet procoagulant response with agents that inhibit the Ca²+-sensitive K⁺ channels, or Gardos channels, preferably while maintaining platelet bleeding arrest function, may be used to treat a variety of disorders.

### CROSS-REFERENCE TO RELATED APPLICATIONS

This application claims priority under 35 U.S.C. § 119(e) to provisional application Ser. No. 60/786,163, filed Mar. 27, 2006, and provisional application Ser. No. 60/794,106, filed Apr. 24, 2006, the contents of both applications are incorporated by reference into the present disclosure in their entirety.

### TECHNICAL FIELD

The present invention relates generally to the field of methods of treatment and methods of identifying drugs generally in the areas of hemostasis, thrombosis, and vascular biology.

### BACKGROUND OF THE INVENTION

Blood platelets fulfill a dual role in the hemostatic process. Their adhesive and aggregating properties affirm the formation of a physical barrier required for the primary arrest of bleeding. In addition, platelets provide a catalytic surface for the assembly of enzyme complexes of the coagulation cascade to ensure an accelerated fibrin formation. The latter function is referred to as the platelet procoagulant response, which involves a remodeling of the platelet plasma membrane.¹ In quiescent platelets, phosphatidylserine (PS) is maintained in the inner leaflet of the plasma membrane mainly through the action of an aminophospholipid translocase.^{2,3} Stimulation of platelets causes aminophospholipid translocase activity to shut down, while simultaneously switching on the activity of a phospholipid scramblase. These events lead within minutes to a collapse of the normal asymmetric lipid distribution, resulting in surface exposure of negatively charged PS required for the binding of coagulation factor complexes. Despite several studies revealing various properties of the scramblase mechanism, the identity of the membrane protein(s) involved remains unresolved.^{2,4,5}

A prerequisite for PS exposure in platelets appears to be a persistent elevation of intracellular Ca²⁺ concentration [Ca²⁺]ᵢ, a condition that is particularly accomplished using a combination of the physiological agonists collagen and thrombin.⁶ A prolonged rise in [Ca²⁺]ᵢ may also cause a collapse of lipid asymmetry in cells other than platelets. In the presence of extracellular Ca²⁺, Ca²⁺-ionophores such as ionomycin or A23187 have been shown to induce PS exposure in a variety of cells, including erythrocytes. Studies by Lang and coworkers⁷ have demonstrated that Ca²⁺, entering erythrocytes, not only activates the scramblase, but also stimulates the Ca²⁺-sensitive “Gardos” K⁺ channel in these cells. Subsequent loss of K⁺ ions together with efflux of Cl⁻ ions induces cell shrinkage, accompanied by shedding of microvesicles from the plasma membrane. It was further demonstrated that these events might be linked as both could be inhibited by either an increase of the extracellular K⁺ concentration or selective Gardos K⁺ channel blockers. Impaired cell shrinkage, microvesicle production and PS exposure upon treatment with Ca²⁺-ionophore are also observed in erythrocytes from patients with Scott syndrome.⁸ This rare hereditary bleeding disorder is caused by a defective scramblase mechanism that appears to be present in all cells of the hematological lineage. Hence, platelets from patients with Scott syndrome exhibit impaired PS exposure and microvesicle production in response to Ca²⁺-ionophore or collagen plus thrombin, despite a normal increase in [Ca²⁺]ᵢ.

### SUMMARY OF THE INVENTION

One aspect of the invention relates to a method of modulating platelet procoagulant response by modulating Ca²⁺-sensitive K⁺ channels of the platelets. Preferably, the modulation is accomplished while maintaining platelet bleeding arrest function. The method of modulating platelet procoagulant response is performed by exposing a platelet to an agent that modulates Ca²⁺-sensitive K⁺ channels of the platelet. In one embodiment, the agent inhibits the Ca²⁺-sensitive K⁺ channels of the platelet Agents useful in inhibiting the Ca²⁺-sensitive K⁺ channels of the platelet include clotrimazol, charybdotoxin, quinine, or biologically active analogs thereof.

In another aspect, the invention relates to a method of modulating platelet procoagulant response by exposing platelets to an elevated extracellular K⁺ concentration. Once again, this is preferably accomplished while maintaining platelet bleeding arrest function.

Another aspect of the invention relates to a method of treating a patient for a disorder involving platelet procoagulant response. In the method, a patient in need of treatment for a disorder involving platelet procoagulant response is selected. An agent that modulates Ca²⁺-sensitive K+ channels of platelets, preferably while also maintaining platelet bleeding arrest function, is administered to the patient in a therapeutically effective amount. Agents useful in the method include clotrimazol, charybdotoxin, quinine, or biologically active analogs thereof.

Disorders involving platelet procoagulant response include acute coronary syndromes, percutaneous intervention, cardiac bypass surgery (CABG), atrial fibrillation, deep vein thrombosis, intermittent claudication, peripheral arterial disease, atherosclerosis, and thrombocytopenia or other bleeding diatheses.

Yet another aspect of the invention is a method of treating a patient with Scott syndrome. The Scott syndrome patient is administered an agent that promotes K⁺ efflux in platelets.

A method of identifying an agent useful for modulating platelet procoagulant response is another aspect of the invention. The identified agent preferably does not interfere with the bleeding arrest capability of platelets.

### BRIEF DESCRIPTION OF THE DRAWINGS

FIG. 1 is a graph illustrating the effect of high extracellular [K⁺] on collagen plus thrombin induced platelet procoagulant response measured as the binding of FITC-conjugated annexin A5.

FIGS. 2A-2B are graphs illustrating the effect of potassium channel inhibitors on collagen plus thrombin induced platelet procoagulant activity analyzed for prothrombinase activity (A) or percentage of annexin-positive cells (B). Black bars: absence of valinomycin; hatched bars: presence of valinomycin.

FIG. 3 is a histogram of the binding of annexin A5 to collagen plus thrombin activated platelets, showing the effect of clotrimazol.

FIG. 4 is a graph of the effect of Gardos channel blockers and valinomycin on collagen plus thrombin induced procoagulant response in HEPES/Choline buffer. Black bars: absence of valinomycin; hatched bars: presence of valinomycin.

FIG. 5 is a graph illustrating the effect of valinomycin on collagen plus thrombin induced procoagulant activity of platelets from a patient with Scott syndrome. Black bars: absence of valinomycin; hatched bars: presence of valinomycin.

### DETAILED DESCRIPTION OF THE PREFERRED EMBODIMENTS

Modulators of the Ca²⁺-sensitive K⁺ channels, or Gardos channels, of platelets have now been found to affect platelet procoagulant activity, which may be used for advantageous purposes such as treating disorders implicated by such platelet procoagulant activity with existing and new modulators.

Selective Gardos channel modulators as well as an increased extracellular K⁺ concentration have been used herein to illustrate the role of the Gardos K⁺ channel in the procoagulant response of platelets to physiological agonists such as the combined action of collagen and thrombin. In addition K⁺ ionophores, such as valinomycin, have been utilized to abolish the effect of the K⁺ channel modulators. In one aspect of the invention, the modulators are inhibitors of the channels and were used to show inhibition of platelet procoagulant response. The Gardos channel inhibitors or blockers, but not other K⁺ channel blockers, inhibited the platelet procoagulant response without having an appreciable effect on other platelet responses involved in the primary arrest of bleeding, such as aggregation or release, pointing out a significant advantage of targeting these channels for therapeutic intervention.

Furthermore, evidence is presented indicating that the impaired PS exposure in Scott syndrome platelets upon activation with collagen and thrombin may be partially attributed to an impaired efflux of K⁺ ions, thus providing a potential treatment for patients with this disorder.

Activation of blood platelets, particularly by the combined action of collagen and thrombin, evokes a procoagulant response, which has been attributed to surface exposure of PS in a distinct platelet population,¹⁷ thus producing a catalytic membrane surface that promotes assembly and activity of the prothrombinase and tenase complex of the blood coagulation proteins.⁹ As demonstrated herein, selective modulation of certain K⁺ channels present in the plasma membrane affects the procoagulant response of platelets.

A number of studies have demonstrated the presence of both voltage operated K⁺ channels and Ca²⁺-induced K⁺ channels in the plasma membrane of platelets.²⁷⁻³⁰ The latter can be subdivided in three different types, SK (small conductance-), IK (intermediate conductance-) and BK (large conductance-) channels.³¹⁻³³ Whether or not all three types are present in platelets is presently unknown. Of the Ca²⁺-activated K⁺ channels, the IK channel is identical to the Gardos channel, first described in erythrocytes.³⁴ Distinction between different types of K⁺ channels can be made based on their sensitivity to specific inhibitors.^{18,32,33} Clotrimazol, an anti-fungal agent, and charybdotoxin, a high affinity peptide isolated from scorpion venom, specifically block the Gardos channel, although charybdotoxin can also inhibit the large-conductance Ca²⁺-activated K⁺ channel. Iberiotoxin, a peptide also purified from scorpion venom and apamin, isolated from bee venom are blockers of BK and SK channels, respectively.^{18,32,33}

### Modulating

Modulating platelet procoagulant response as taught herein includes inhibition as well as stimulation of the Ca²⁺-sensitive K⁺ channels of the platelets. If desired, inhibition of the channel may be reversed with a K+ ionophore, such as valinomycin or nigericin.

The terms “inhibit,” “inhibiting” and “inhibition” as used herein encompass fully or partially inhibiting the function of the channels. In a preferred embodiment, the inhibition is at least 20% inhibition, preferably, at least 30% inhibition, more preferably at least 40% inhibition, more preferably at least 50% inhibition, more preferably at least 60% inhibition, more preferably at least 70% inhibition, more preferably 80% inhibition, even more preferably at least 90% inhibition, most preferably, the inhibition is at 100%. Inhibition may be measured by a reduction in efflux of K⁺ as compared to baseline.

The terms “stimulate” or “stimulating” include even minimal agonistic effect and preferably at least 10% greater effect as compared to baseline, more preferably at least 20% greater effect as compared to baseline, more preferably at least 30% greater effect as compared to baseline, more preferably at least 40% greater effect as compared to baseline, more preferably at least 50% greater effect as compared to baseline, more preferably at least 60% greater effect as compared to baseline, even more preferably at least 70% greater effect as compared to baseline, and most preferably 80% or more of a greater effect as compared to baseline. Stimulation may be measured by an increase in efflux of K⁺ as compared to baseline.

The term “minimally” when describing a modulator that “interferes minimally with the bleeding arrest” capabilities of the patient includes modulators with less than 25% interference of bleeding arrest function, more preferably less than 10% interference, and most preferably less than 5% interference.

### Modulators of the Ca²⁺-Sensitive K⁺ Channels

Modulators of the Ca²⁺-sensitive K⁺ channels include the compounds clotrimazol (also known as clotrimazole), charybdotoxin, and quinine, as well as biologically active analogs thereof.

“Analogs”, as used herein, includes derivatives of the compounds as well as structural and/or functional analogs.

“Biologically active analog” refers to an analog characterized by having at least one of the biological activities described herein. The biological activity of an analog can be determined, for example, as described in the Examples Section. In one embodiment, the biologically activity is modulation of Ca²⁺-sensitive K⁺ channels. In an alternative preferred embodiment, the biologically activity is reversing inhibition of Ca²⁺-sensitive K⁺ channels.

Clotrimazol is a triarylmethane of the formula 1-[(2-chlorophenyl)diphenylmethyl]-1H-imidazole. Examples of analogs of clotrimazol include, but are not limited to, TRAM-34 ([1-(2-chlorophenyl)diphenyl)methyl]-1H-pyrazole), TRAM-3 ((2-chlorophenyl)diphenylmethanol), and TRAM-39 (2-(2-chlorophenyl)-2,2-diphenylacetonitrile (Wulff, H., et al., (2001) J. Biol. Chem., 276 (34): 32040-32045, incorporated by reference herein) and ICA 17043 (Icagen) (2,2-bis(4-fluorphenyl)-2-phenylacetamide) (Stocker, J. W., et al, Blood (2003) 101 (6):2412-2418, both of which are incorporated by reference herein).

Charybdotoxin is a well-characterized polypeptide neurotoxin present in the venom of the scorpion Leiurus quinquestriatus hebraeus that blocks calcium-activated potassium channels. See for example, Miller, C. et al., (1985) Nature 313:316-318, Rauer, H., et al, (2000) J. Biol Chem, 275 (2): 1201-1208; Vazquez, J. et al. (1990), J. Biol. Chem. 265:15564-15571; Bontems, F. et al. (1991), Science 254: 1521-1523; Park, C. S. et al. (1991) PNAS 88:2046-2050; Vita, C. et al. (1993) Eur. J. Biochem. 217: 157-169; Massefski Jr, W. et al., (1990) Science 249:521-524; Vita, C., et al., (1995) PNAS 92:6404-6408; all of which are incorporated by reference herein.

Analogs of charybdotoxin can be generated using any technique known in the art including genetically engineering the polypeptide or generating analogs using synthetic peptide synthesis techniques. Possible analogs include charybdotoxin polypeptides with one or more amino acid deletion, addition, or substitution as compared to wildtype charybdotoxin. Such analogs exhibit at least 80%, more preferably at least 90% homology, even more preferably at least 100% homology with charybdotoxin.

Charybdotoxin polypeptide analogs also include modified polypeptides. Modifications of polypeptides of the invention include chemical and/or enzymatic derivatizations at one or more constituent amino acid, including side chain modifications, backbone modifications, and N- and C-terminal modifications including acetylation, hydroxylation, methylation, amidation, and the attachment of carbohydrate or lipid moieties, cofactors, and the like.

### Screening

The methods of the invention may be utilized to identify new agents for modulating platelet procoagulant response. As presented in the examples, clotrimazol and charybdotoxin are useful modulators of the Ca²⁺-sensitive K⁺ channels of platelets, especially as they have minimal impact on the platelet bleeding arrest function. Quinine may also be a useful modulator in certain circumstances. Biologically active analogs of clotrimazol, charybdotoxin, and quinine, or of other newly identified modulators of the Ca²⁺-sensitive K⁺ channels of platelets, that have similar functional characteristics to these agents may be utilized in the methods taught herein.

Furthermore, clotrimazol, charybdotoxin, quinine, as well as biologically active analogs thereof, may be utilized as reference compounds for identifying new modulators. A screening method may include exposing Ca2+-sensitive K+ channels of platelets to a candidate agent, whether novel or known, or libraries of candidate agents.

Accordingly, one aspect of the invention provides for a method of identifying an agent useful for modulating platelet procoagulant response. In one embodiment, the method of identifying an agent useful for modulating platelet procoagulant response is performed by exposing Ca2+-sensitive K+ channels of platelets to a candidate agent and determining the modulatory effect of the candidate agent on the channels.

In a preferred embodiment, the modulatory effect of the candidate agent is determined by comparing the K⁺ efflux through the Ca²⁺-sensitive K⁺ channels in the presence of the candidate agent to the K⁺ efflux through the Ca²⁺-sensitive K⁺ channels in the absence of the candidate agent. In this embodiment, a candidate agent will be selected as a useful agent for modulating platelet procoagulant response if the Ca²⁺-sensitive K⁺ channels are modulated, either through inhibition or stimulation, in the presence of the candidate agent at a level greater than that observed in the absence of the candidate agent. Such candidates may be chosen for further characterization as therapeutic agents.

In a further preferred embodiment, the modulatory effect of the candidate agent is determined by comparing the K⁺ efflux through the Ca²⁺-sensitive K⁺ channels in the presence of the candidate agent to the K⁺ efflux through the Ca²⁺-sensitive K⁺ channels in the presence of at least one reference compound.

In the case of clotrimazol, charybdotoxin, quinine, and biologically active analogs thereof, serving as reference compounds, the modulatory effect of the candidate agent and the reference compound is likely to be a comparison of inhibitory capabilities. One may identify stimulatory modulators, however, using generally inhibitory reference compounds or may utilize stimulatory modulators as reference compounds to identify further stimulatory modulators.

A candidate agent will be selected as a useful agent for modulating platelet procoagulant response if its modulatory effect compares favorably with the reference compound such as by providing evidence of the same or greater modulatory effect, e.g. inhibitory activity. A candidate agent can also be considered a useful agent for modulating platelet procoagulant response if it has certain desirable properties, such as a high level of selectivity and/or has a more desirable performance as a drug candidate. For example, a candidate agent is useful if it has a reduced number or a reduced intensity of undesirable side effects. As noted, useful agents include candidate agents that provide evidence of stimulatory activity, which may be measurable as a relative level of change of modulation in the opposite direction.

Ideally, any candidate agent selected for further investigation or use will have minimal impact on the platelet bleeding arrest function of the platelets. This may be tested as presented in example 5 below.

### Treatment

Modulation of the Ca²⁺-sensitive K⁺ channels of platelets is a useful mode of treating disorders involving the platelet procoagulant response. In many cases, an inhibition of the channels is most appropriate for treatment. A patient may be beneficially treated by administration of a modulator of the channel. Treatment according to the invention may constitute administration of one or more inhibitors of the Ca²⁺-sensitive K⁺ channels, such as those disclosed herein, or biologically active analogs thereof.

In particular, the use of clotrimazol, charybdotoxin, quinine, or biologically active analogs thereof, to treat a patient for a disorder involving platelet procoagulant response is within the scope of the invention.

The use of modulators of Ca²⁺-sensitive K⁺ channels, such as clotrimazol, charybdotoxin, or quinine, or biologically active analogs thereof, in the manufacture a medicament for treatment of a patient for a disorder involving platelet procoagulant response is also taught herein.

The methods uses, and compositions taught herein are useful for treating patients with a need for such channel modulators (e.g., those suffering from disorders involving the platelet procoagulant response). More particularly, the methods of the invention are useful in conjunction with treatment of disorders of the circulatory and cardiovascular systems. Disorders treatable by such modulators of Ca²⁺-sensitive K⁺ channels include acute coronary syndromes, percutaneous intervention, cardiac bypass surgery (CABG), atrial fibrillation, deep vein thrombosis, intermittent claudication, peripheral arterial disease, atherosclerosis, and thrombocytopenia or other bleeding diatheses.

Preferably, a patient is selected for treatment based on their ability to benefit from the administration of Ca²⁺-sensitive K⁺ channels modulators. These patients exhibit or suffer from disorders involving the platelet procoagulant response, including disorders of the circulatory and/or cardiovascular systems as discussed above.

Administration in vivo can be effected in one dose, continuously or intermittently throughout the course of treatment. Methods of determining the most effective means and dosage of administration are well known to those of skill in the art and will vary with the composition used for therapy, the purpose of the therapy, and the subject being treated. Single or multiple administrations can be carried out with the dose level and pattern being selected by the treating physician. Suitable dosage formulations and methods of administering the agents may be empirically adjusted.

More particularly, an agent administered according to the invention may be administered for therapy by any suitable route including oral, rectal, nasal, topical (including transdermal, aerosol, buccal and sublingual), vaginal, parenteral (including subcutaneous, intramuscular, intravenous and intradermal), and pulmonary. It will also be appreciated that the preferred route will vary with the condition and age of the recipient and the disorder being treated.

Ideally, the agent should be administered to achieve peak concentrations of the active compound at sites of the disorder or disease. This may be achieved, for example, by the intravenous injection of the agent, optionally in saline or orally administered, for example, as a tablet, capsule or syrup containing the active ingredient. Desirable blood levels of the agent may be maintained by a continuous infusion to provide a therapeutic amount of the active ingredient within a disease tissue. In a specific embodiment, it may be desirable to administer pharmaceutical compositions locally to the area in need of treatment; this may be achieved by, for example, and not by way of limitation, local infusion during surgery, by injection, or by means of a catheter. The use of operative combinations may provide therapeutic combinations requiring a lower total dosage of each component agent than may be required when each individual therapeutic compound or drug is used alone, thereby reducing adverse effects.

While it is possible for the agent to be administered alone, it is preferable to present it as a pharmaceutical formulation comprising at least one active ingredient together with one or more pharmaceutically acceptable carriers and optionally other therapeutic agents. Each carrier must be “acceptable” in the sense of being compatible with the other ingredients of the formulation and not injurious to the patient.

Pharmaceutical compositions utilized according to the methods of the invention may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to the key active ingredients, the pharmaceutical compositions can also contain other pharmaceutically active compounds or a plurality of compositions of the invention.

Formulations also include those suitable for oral, rectal, nasal, topical (including transdermal, buccal and sublingual), vaginal, parenteral (including subcutaneous, intramuscular, intravenous and intradermal) and pulmonary administration. The formulations may conveniently be presented in unit dosage form and may be prepared by any methods well known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both and then, if necessary, shaping the product.

Formulations suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin or sucrose and acacia; and mouthwashes comprising the active ingredient in a suitable liquid carrier.

Pharmaceutical compositions or formulations for topical administration according to the present invention may be formulated as an ointment, cream, suspension, lotion, powder, solution, paste, gel, spray, aerosol, or oil. Alternatively, a formulation may comprise a patch or a dressing such as a bandage or adhesive plaster impregnated with active ingredients and optionally one or more excipients or diluents.

If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the agent through the skin or other affected areas. Examples of such dermal penetration enhancers include dimethylsulfoxide and related analogues.

The oily phase of the emulsions of a composition used according to this invention may be constituted from known ingredients in a known manner. While this phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulphate.

The choice of suitable oils or fats for the formulation is based on achieving the desired cosmetic properties, since the solubility of the active compound in most oils likely to be used in pharmaceutical emulsion formulations is very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with a suitable base comprising, for example, cocoa butter or a salicylate.

Formulations suitable for vaginal administration may be presented as pessaries, tampons, creams, gels, pastes, foams, or spray formulations containing in addition to the agent, such carriers as are known in the art to be appropriate.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the agent.

Formulations suitable for parenteral administration include aqueous and non-aqueous isotonic sterile injection solutions that may contain anti-oxidants, buffers, bacteriostats, and solutes which render the formulation isotonic with the blood of the intended recipient. Other suitable formulations may be aqueous and non-aqueous sterile suspensions that may include suspending agents, thickening agents, and liposomes or other microparticulate systems that are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets of the kind previously described.

As noted, various delivery systems are known and can be used to administer a therapeutic agent in accordance with the methods of the invention, e.g., encapsulation in liposomes, microparticles, or microcapsules. The compounds can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq. (1976).

Preferred unit dosage formulations are those containing a daily dose or unit, daily subdose, as herein above-recited, or an appropriate fraction thereof, of an agent. Therapeutically effective amounts of the compounds generally include any amount sufficient to detectably modulate the Ca²⁺-sensitive K⁺ channels by standardized assays or experimentation or by detecting an alleviation of symptoms of the disorder.

The amount of active ingredient that may be combined with the carrier materials to produce a single dosage form will vary depending upon the subject treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disorder for which the patient is undergoing therapy. The therapeutically effective amount for a given situation can be readily determined by routine experimentation and is within the skill and judgment of the ordinary clinician.

For purposes of the present invention, a therapeutically effective dose will generally be a total daily dose administered to a host in single or divided doses and may be in amounts, for example, of from 0.001 to 1000 mg/kg body weight daily and more preferred from 1.0 to 30 mg/kg body weight daily. Dosage unit compositions may contain such amounts of submultiples thereof to make up the daily dose.

Therapeutic agents utilized according to this invention include, but are not limited to, small molecules. They may be polynucleotides, peptides, antibodies, antigen presenting cells and include immune effector cells that specifically recognize and act upon cells expressing the gene of interest. One can determine if a subject or patient will be beneficially treated by the use of agents by screening one or more of the agents against platelets isolated from the subject or patient using methods known in the art.

Alternatively, small molecule inhibitors may be used in combination with other types of treatments. For instance, inhibitors that are not small molecules, e.g. biologicals, polynucleotides, gene therapy, etc. may be used in conjunction in a combination protocol for treatment.

As noted, while the agents useful in the methods of the invention can be administered as the sole active pharmaceutical agent, they can also be used in combination with one or more other agents to treat a disorder. Thus, the agents are also useful in combination with known therapeutic agents, and combinations with other therapeutic agents are within the scope of the invention. Therapeutic agents especially useful in combination with the Ca²⁺-sensitive K⁺ channel modulators include aspirin, a TP antagonist (thromboxane receptor antagonist), thromboxane synthase inhibitor, a P2Y12 antagonist (purinergic receptor P2Y, G-protein coupled 12), syk (spleen tyrosine kinase) kinase inhibitor, and dipyrimadole. The health care provider should be able to discern which combinations of agents would be useful based on the particular characteristics of the agents and the disorder involved.

Compounds to be employed in combination with the agents for channel modulation may be used in therapeutic amounts as indicated in the Physicians' Desk Reference (PDR) 60th Edition (2006), which is incorporated herein by reference, or such therapeutically useful amounts as would be known to one of ordinary skill in the art.

The agents and secondary compounds may be administered at the recommended maximum clinical dosage or at lower doses, within the judgment of the treating physician. Dosage levels of the active compounds in the compositions of the invention may be varied so as to obtain a desired therapeutic response depending on the route of administration, severity of the disease, and the response of the patient. The combination can be administered as separate compositions or as a single dosage form containing both agents. When administered as a combination, the therapeutic agents can be formulated as separate compositions, which are given at the same time or different times, or the therapeutic agents, can be given as a single composition. Combination therapy includes sequential administration of therapeutic agents as well as co-administration of such agents.

As taught herein, modulation of the platelet procoagulant response, preferably while maintaining platelet bleeding arrest function, may also be accomplished by exposing platelets to an elevated extracellular K⁺ concentration. Agents that allow for an elevated extracellular K⁺ concentration are useful in the methods of modulation in the same manner as the inhibitors discussed above. Since elevating such concentration in the instance of treating a subject may have other, possibly unintended or undesired, effects, care must be taken in exposing platelets to an elevated extracellular K⁺ concentration in an appropriate manner such as with an appropriate formulation.

Modulation does not only include inhibition of the Gardos channels of platelets, but may also include stimulation in certain circumstances. In the case of patients with Scott syndrome, stimulation, such as by exposure to the patient or his platelets to an agent that promotes K⁺ efflux in platelets may be an effective course of action.

### EXAMPLES

The invention is explained in detail by the working examples presented herein.

The examples below as well as throughout the application, the following abbreviations have the following meanings. If not defined, the terms have their generally accepted meanings.

ATP=adenosine triphosphate

BSA=bovine serum albumin

g=Gram

HEPES=4-(2-hydroxyethyl)-1-piperazineethanesulfonic acid

M=Molar

mg=milligram

MHz=megahertz

mL=milliliter

mM=millimolar

mmol=millimole

nM=nanomolar

nmol=nanomole

nm=nanometer

rpm=revolutions per minute

SD=standard deviation

μL=microliters

### Material and Methods

### Reagents

Calcium ionophore ionomycin, bovine serum albumin (BSA, essentially fatty acid free), clotrimazol, quinine hydrochloride, charybdotoxin, apamin, iberiotoxin, and valinomycin were obtained from Sigma (St Louis, Mo.). Human thrombin was purified as previously described⁹ and collagen was from Horm (Nycomed, Münich, Germany). Tirofiban was obtained from MSD (Haarlem, the Netherlands). The coagulation proteins prothrombin, factor Xa and factor Va were purified from bovine blood as described before.⁹ Thrombin-specific chromogenic substrate S2238 was obtained from Chromogenix (Mölndal, Sweden). Fluorescein isothiocyanate (FITC)-conjugated annexin A5 and Fura-2 were from Invitrogen (Leiden, the Netherlands). ATP Bioluminescence Assay Kit HS II was from Boehringer Mannheim (Germany).

### Platelet Isolation and Activation

Platelet isolation was performed as described.¹⁰ Briefly, 10 milliliters (mL) blood was collected in 2 mL anticoagulant ACD (80 mmol/L trisodium citrate, 52 mmol/L citric acid and 180 mmol/L glucose). Platelet rich plasma was obtained by centrifuging whole blood for 15 minutes at 200 g. Platelets were spun down at 11,500× gravity for 2 minutes using a microfuge. The platelet pellet was resuspended and washed twice with 10 mmol/L HEPES, 137 mmol/L NaCl, 2.7 mmol/L KCl, 2 mmol/L MgCl₂, 5 mmol/L glucose and 0.5 mg/mL BSA, adjusted at pH 6.6); before each centrifugation step, 100 μL ACD was added to each mL resuspended platelets. Finally, platelets were resuspended in HEPES buffer at pH 7.4. Platelet count was adjusted at 2×10⁸ platelets/mL. For measurement of the intracellular Ca²⁺ concentration ([Ca²⁺]ᵢ), platelets were loaded with Fura-2 (3 μmol/L) during 45 minutes at 37° C., prior to isolation.

Platelet activation was performed at 37° C. at a concentration of 10⁷ platelets/mL in a volume of 0.5 mL under continuous stirring in the presence of 3 mmol/L CaCl₂. Platelet agonists used were thrombin (5 nmol/L) plus collagen (10 μg/mL) or ionomycin (1 μmol/L). The effect of different inhibitors was studied by pre-treatment of platelets with the various agents for 30 minutes before starting the activation procedure. For the effects of high extracellular [K⁺], platelet suspensions were diluted 20-fold to a concentration of 107 platelets/mL in a modified HEPES buffer, referred to as HEPES/KCl buffer (10 mmol/L HEPES, 19.7 mmol/L NaCl, 120 mmol/L KCl, 2 mmol/L MgCl₂, 5 mmol/L glucose and 0.5 mg/mL BSA, pH 7.4), activated and assayed for thrombin generation as described before. In some experiments, HEPES buffer was used in which 120 mmol/L NaCl was replaced by 120 mmol/L choline chloride (10 mmol/L HEPES, 17 mmol/L NaCl, 120 mmol/L choline chloride, 2.7 mmol/L KCl, 2 mmol/L MgCl₂, 5 mmol/L glucose and 0.5 mg/mL BSA, pH 7.4), referred to as HEPES/Choline buffer.

For experiments with platelets from a patient with Scott syndrome, freshly obtained citrated blood from the patient and a control was shipped by airmail and platelets were isolated and used for experiments immediately upon arrival (approx 24 hours after blood donation).

### Platelet Prothrombinase Activity

Platelet procoagulant activity was assayed as described in more detail elsewhere.⁹ Briefly, after activation of 10⁷ platelets/mL with collagen (10 μg/mL) and thrombin (4 nmol/L) for 10 minutes, samples were taken and diluted to 10⁶ platelets/mL in HEPES buffer containing 3 mmol/L CaCl₂ and 0.5 mg/mL BSA. The samples were incubated with 0.2 nmol/L factor Xa and 2 nmol/L factor Va for 1 minute at 37° C. Thrombin formation was initiated by addition of 1 μmol/L prothrombin and arrested after 2 minutes by addition of 10 mmol/L EDTA. Thrombin was measured using the chromogenic substrate S2238.

Separation of Microparticles from Remnant Platelets after Activation

Platelet activation has been demonstrated to be accompanied by shedding of microparticles from the plasma membrane.^{11,12} To evaluate the contribution of microparticles, we have used a previously described method to separate microparticles from remnant cells.¹³ Briefly, after activation, platelet suspensions were centrifuged at 11,500 g; both the prothrombinase activity remaining in the supernatant and the lipid content of the supernatant approached constant values after 3 minutes centrifugation. This prothrombinase activity is operationally defined as derived from microparticles. The absence of platelets or remnant cells in the supernatant was confirmed by flow cytometry.

### Flow Cytometry

Samples for flow cytometry were taken simultaneously with samples for the prothrombinase assay from the platelet incubations after 10 minutes activation. To measure PS exposure, samples were diluted 10-fold in HEPES buffer containing 3 mmol/L CaCl₂ and incubated with FITC-conjugated Annexin A5 (final concentration 10 nmol/L) for 5 minutes and analyzed for light scatter and fluorescence by a Coulter Epics XL-MLC™ flow cytometer. Light scatter and fluorescence channels were set at logarithmic gain. Scatter parameters and fluorescence intensities were obtained from 10,000 platelets and analyzed off-line using WinMDI version 2.8 software (http://facs.scripps.edu/software.html). Ionomycin activated platelets were used to set a marker for fluorescence intensity of annexin-positive platelets to measure the percentage of annexin-positive platelets after the various incubation procedures.

### Measurement of [Ca²⁺]ᵢ of Platelets in Suspension

Platelets (5×10⁷/mL) were incubated with K⁺ channel inhibitors for 5 minutes, followed by the addition of tirofiban (5 μg/mL) and Ca²⁺ (3 mmol/L) and incubated for an additional 5 minutes under stirring conditions. Collagen (10 μg/mL) plus thrombin (5 nmol/L) were then added simultaneously. Changes in [Ca²⁺]ᵢ of Fura-2-loaded platelets were measured under continuous stirring by dual excitation fluorometry in an SLM-Aminco 8100 spectrofluorimeter (SLM Instruments). Ratio values of fluorescence at 340 and 380 nm excitation, obtained 5 minutes after activation with collagen plus thrombin, were converted to levels of [Ca²⁺]ᵢ as described.¹⁴ Experiments were performed in triplicate at 37° C.

### Measurement of Platelet Aggregation and Release

Aggregation was measured at 37° C. in 450 μL samples of a platelet suspension of 2×10⁸/mL using an automated model 400 Chronolog aggregometer (Chronolog Corporation, Chicago, USA). After stirring for 2 minutes at 300 rpm, CaCl₂ (3 mmol/L) was added, 1 minute later followed by simultaneous addition of 10 μg/mL collagen and 4 nmol/L thrombin. The resulting aggregation was recorded for 5 minutes after which 50 μL ATP-reagent (Boehringer Mannheim ATP Bioluminescence Assay Kit HS II) was added to quantify the release reaction.

### Statistics

Data are expressed as mean values±1 SD. To determine the statistical significance of differences, P-values were calculated by Student's t-test (*P<0.05; **P<0.01; ***P<0.001)

### Example 1

### The Influence of High Extracellular [K⁺] on the Generation of Procoagulant Activity

Initial experiments to demonstrate the importance of an efflux of K⁺ ions during platelet stimulation on the procoagulant response were designed by changing the cation composition of the buffer. In these experiments, high extracellular [K⁺] in the HEPES/KCl buffer was compensated by reducing [Na⁺] in order to maintain osmolarity. It should be emphasized, however, that a decreased [Na⁺] may have two major consequences: it reduces the catalytic efficiency of thrombin¹⁵ and it likely causes inhibition of the Na⁺/H⁺ exchanger, which may be involved in PS exposure during platelet activation.¹⁶ We therefore compared the procoagulant response induced by collagen plus thrombin in low [Na⁺] solutions (i.e., HEPES/Choline buffer and HEPES/KCl buffer) with the response evoked in high [Na⁺] solution (i.e., HEPES buffer). In these initial experiments, the procoagulant response was determined by the number of annexin binding platelets measured by flow cytometry.

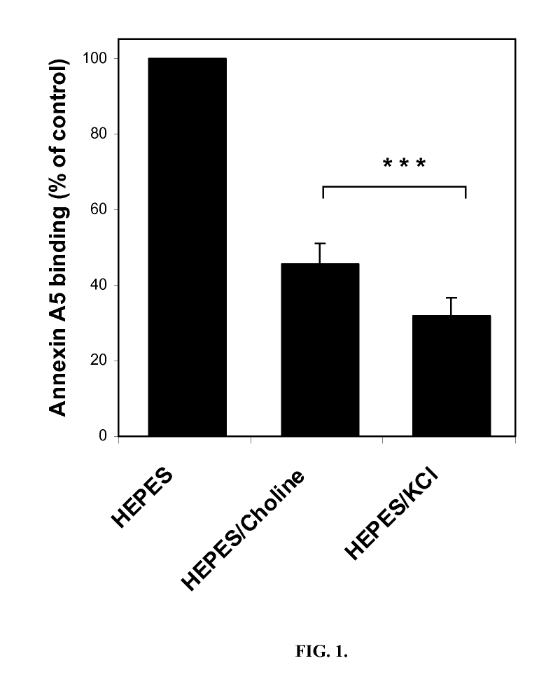

FIG. 1. shows the effect of high extracellular [K⁺] on collagen plus thrombin induced platelet procoagulant response. Platelets at 10⁷ ml⁻¹ were activated with collagen (10 μg/ml) plus thrombin (4 nmol/L) for 10 minutes. in HEPES, HEPES/Choline, or HEPES/KCl and the procoagulant response was measured as the binding of FITC-conjugated annexin A5. Data are expressed as a percentage of the response obtained in HEPES. The 100% value in HEPES corresponds to approx. 20-25% of the total cell population which becomes positive for annexin binding, depending on the donor. Data are mean values±1 S.D. (n=4).

As shown in FIG. 1, the response in HEPES/Choline buffer decreased to 45.3±5.6% (n=4) of that induced in HEPES buffer, whereas in HEPES/KCl buffer the response dropped to 31.9±4.8% (n=4). Thus, although the low [Na⁺] caused a decreased procoagulant response in both buffers, there appeared to be a significant additional inhibitory effect of the presence of high extracellular K⁺, supporting the notion that efflux of K⁺ ions contributes to the procoagulant response.

### Example 2

### The influence of High Extracellular [K⁺] on Microvesicle Formation

Since microvesicle formation and PS exposure are closely associated events in the procoagulant response¹¹⁻¹³, we also studied the effect of a high [K⁺] on microvesicle formation in collagen and thrombin activated platelets. Because of their small size, flow cytometry is not particularly suitable for quantifying platelet microparticles. Therefore, formation of microvesicles was operationally defined as residual procoagulant activity in the supernatant of a platelet suspension that was centrifuged at 11,500×g for 3 minutes as was described previously.^{13,17} In HEPES/KCl, a 50% reduction in prothrombinase activity of both the supernatant and the total platelet incubation was found, indicating that a reduced PS exposure was accompanied by a reduced formation of microvesicles.

### Example 3

### Gardos Channel Blockers Inhibit Platelet Procoagulant Response

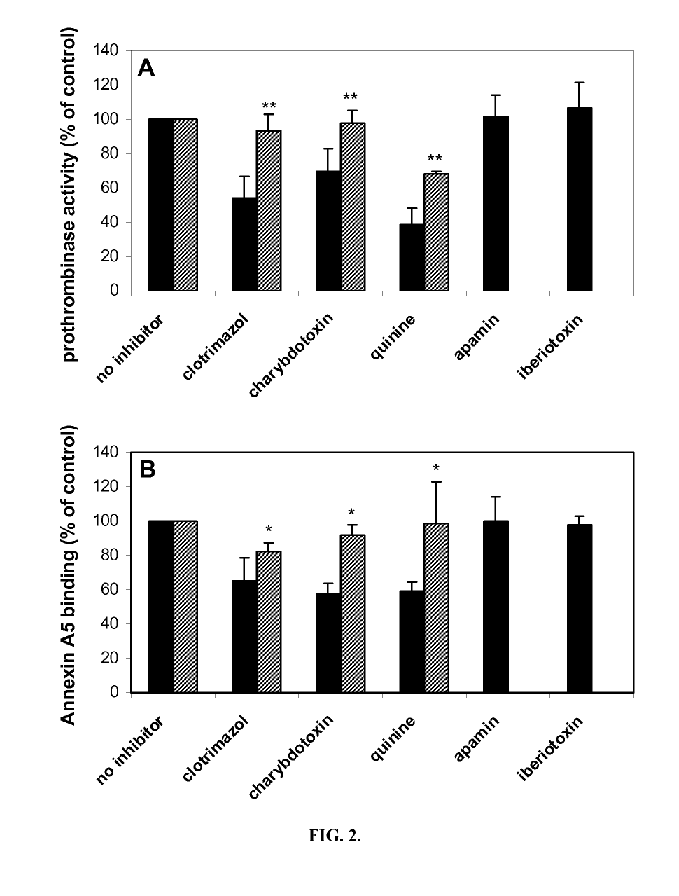

Various K⁺ channel inhibitors have been tested for their ability to affect the collagen plus thrombin-induced platelet prothrombinase activity. The results are depicted in FIG. 2A.

FIG. 2. illustrates the effect of potassium channel inhibitors on collagen plus thrombin induced platelet procoagulant activity. Platelets at 10⁷ ml⁻¹ were preincubated with various inhibitors for 30 minutes followed by activation with collagen (10 μg/ml) plus thrombin (4 nmol/L) for 10 minutes. Samples from these incubations were diluted 10-fold in HEPES buffer containing 3 mmol/L CaCl₂ and subsequently analyzed for prothrombinase activity (A) or percentage of annexin-positive cells (B) as described in the Materials and Methods section above. Valinomycin was added 5 minutes prior to activation of the platelets (to avoid long term effects of this ionophore). Black bars: absence of valinomycin; hatched bars: presence of valinomycin. Concentrations used: clotrimazol, 10 μmol/L; charybdotoxin, 20 nmol/L; quinine, 0.4 mmol/L; apamin, 500 nmol/L; iberiotoxin, 200 nmol/L; valinomycin, 3 μmol/L. Data are expressed as percentage of control, i.e. platelets in absence of inhibitor (mean±1 S.D. A: n>6; B: n>3). Student's t-test was used to compare data in the presence and absence of valinomycin.

The Gardos channel inhibitors clotrimazol (10 μmol/L) and charybdotoxin (20 nmol/L) caused 46±13% (n=12) and 31±14% (n=9) inhibition of the prothrombinase activity of collagen and thrombin activated platelets, respectively. The estimated IC₅₀ for these two inhibitors on the platelet procoagulant response are 100 nmol/L and 5 nmol/L, respectively, in good agreement with the estimated potencies to inhibit K⁺ fluxes.¹⁸ Quinine, one of the first compounds demonstrated to block K⁺ efflux in erythrocytes¹⁹⁻²¹, appeared to be the most potent inhibitor, being able to decrease the collagen plus thrombin-induced procoagulant activity by 60±10% (n=6). Two other K⁺ channel inhibitors, iberiotoxin and apamin, were without effect. Very similar findings with these inhibitors were obtained when the procoagulant response was measured as the binding of annexin A5 (FIG. 2B).

To investigate whether inhibition of the procoagulant response by clotrimazol, charybdotoxin and quinine was caused by a preferential effect of these inhibitors on the process of microvesicle formation, we compared the prothrombinase activity of a total suspension of activated platelets with that of its corresponding 11,500×g supernatant. For each inhibitor, the decrease in prothrombinase activity of the supernatant was similar to that of the total platelet suspension, i.e. the prothrombinase activity of the supernatant varied between 30-35% of that of the total suspension, irrespective of the presence of an inhibitor.

### Example 4

### Gardos Channel Blockers Decrease the Fraction of PS-Exposing Platelets after Collagen Plus Thrombin Stimulation

It has been demonstrated that the procoagulant activity of platelets in suspension is caused by a fraction of the platelets showing maximal PS exposure.^{6,17,22,23} The level of procoagulant activity induced by various agonists is determined by the size of the subpopulation of platelets that have maximal externalized PS. To investigate whether the inhibition of procoagulant activity by the K⁺ channel blockers is caused by a decrease in the fraction of PS-exposing platelets rather than by a decrease in the extent of PS exposure of the procoagulant platelet fraction, we used flow cytometry to measure the binding of FITC-conjugated annexin A5 on single cell level.

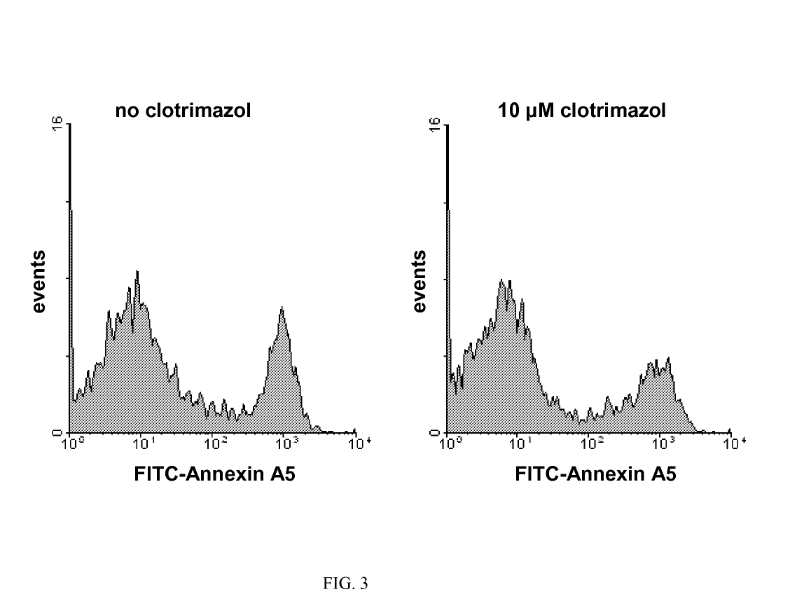

FIG. 3. is a histogram of the binding of annexin A5 to collagen plus thrombin activated platelets showing the effect of clotrimazol. Platelets were pre-incubated with (right histogram) or without (left histogram) clotrimazol (10 μmol/L) prior to activation with collagen plus thrombin. Following activation, platelet samples were diluted and FITC-conjugated annexin A5 was added.

The results shown in FIG. 3 illustrate a typical example of the effect of a K⁺ channel inhibitor, clotrimazol, on the number of annexin-positive platelets after activation with collagen plus thrombin. The number of PS-exposing platelets is decreased in the presence of clotrimazol, but the extent of annexin binding (mean fluorescence intensity) of the residual PS positive platelets remains at the same level as found in the absence of inhibitor. Similar results were obtained for charybdotoxin- or quinine-treated platelets.

Besides a role in supporting blood coagulation, surface exposed PS is a hallmark of the apoptotic process, serving as a signal for phagocyting cells. As shown previously, local anesthetics can cause PS exposure in platelets, a phenomenon associated with mitochondrial-related apoptotic-like events.^{17,24} In our experiment, neither clotrimazol nor charybdotoxin could inhibit tetracaine-induced prothrombinase activity or binding of FITC-conjugated annexin A5.

### Example 5

### No Effect of Gardos Channel Inhibitors on Collagen Plus Thrombin Induced Aggregation and Release

To investigate whether or not other platelet functions were affected by the presence of K⁺ channel inhibitors under these conditions of platelet stimulation, we studied their effect on aggregation and release of ATP. Neither high extracellular [K⁺], nor the presence of clotrimazol or charybdotoxin showed an appreciable effect on aggregation or release (Table 1). (The diminished ATP release observed for platelets in HEPES/KCl appeared to be an apparent decrease, caused by a reduced activity of the ATP reagent (luciferine/luciferase) in this buffer). Quinine caused approximately 60% inhibition of the aggregation, but had only a minor effect on the release. It should be noted in this respect, that quinine has also been observed to inhibit phospholipase A₂, which may result in a decreased thromboxane A₂ production, a secondary stimulator of platelet aggregation.²⁵ Since we were exclusively interested in inhibitory effects under conditions of platelet stimulation that evoke a procoagulant response, we did not perform dose-response curves for each activator separately. Hence, it cannot be excluded that these K⁺ channel blockers may affect the aggregation and/or release induced by collagen or thrombin at lower dose.

TABLE 1
Effect of Gardos Channel Inhibitors
on Platelet Aggregation and Release

| | Aggregation | Release (ATP) |
|---|---|---|
| | % of control (±S.D.) | % of control (±S.D.) |
| High K⁺ (120 mmol/L) | 100.8 ± 6.0 | 79.4 ± 3.3 |
| Clotrimazol (10 μmol/L) | 90.0 ± 2.8 | 91.3 ± 5.0 |
| Charybdotoxin (20 nmol/L) | 108.3 ± 10.2 | 98.7 ± 9.0 |
| Quinine (0.4 mmol/L) | 40.0 ± 3.0 | 90.1 ± 4.1 |

Thus, the Gardos-channel inhibitors used herein do not affect platelet aggregation and release to any appreciable extent. This opens new perspectives to explore selective inhibition of the platelet procoagulant response while maintaining the platelet functions required for the primary arrest of bleeding.

### Example 6

### Inhibition of the Procoagulant Response by Gardos Channel Blockers is Reversed by Valinomycin

To verify that the inhibition by clotrimazol, charybdotoxin, and quinine was caused by blocking the efflux of K⁺ ions from the cell, valinomycin was used, which acts as a cage carrier selective for K⁺ ions. As shown in FIGS. 2A and B (hatched bars), valinomycin at a concentration of 3 μmol/L completely reversed the inhibition caused by clotrimazol and charybdotoxin, and partially abolished the inhibitory effect of quinine. The results depicted in FIG. 2 were from experiments performed in HEPES buffer.

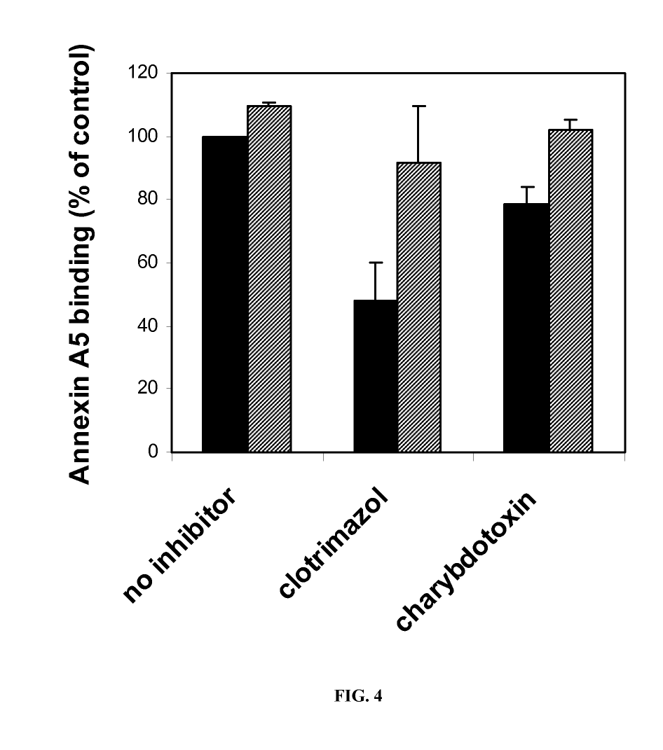

Similar findings were obtained when the experiments were performed in HEPES/Choline. Moreover, also in HEPES/Choline, the inhibition by clotrimazol and charybdotoxin was virtually annihilated in the presence of valinomycin (FIG. 4).

The graph in FIG. 4. illustrates the effects of Gardos channel blockers and valinomycin on collagen plus thrombin induced procoagulant response in HEPES/Choline buffer. Platelets at 10⁷ ml⁻¹ in HEPES/Choline were preincubated with inhibitors for 30 minutes followed by activation with collagen (10 μg/ml) plus thrombin (4 nmol/L) for 10 minutes. Valinomycin was added 5 minutes prior to activation. Samples from these incubations were diluted 10-fold and subsequently analyzed for binding of FITC-conjugated annexin A5. Data are expressed as a percentage of the number of annexin positive cells found upon activation in HEPES/Choline in absence of the inhibitors. Data are mean values±1 S.D. (n=4).

Valinomycin itself did not cause a procoagulant response of platelets in the absence of a stimulus, nor did it affect the procoagulant response by collagen and thrombin in the absence of inhibitors. The inhibition of the procoagulant response by high extracellular [K⁺] was not reversed by addition of valinomycin (data not shown). Collectively, these findings strongly indicate that an efflux of K⁺ ions through the Gardos channel augments the platelet procoagulant response induced by collagen plus thrombin.

### Example 7

### Effect of Gardos Channel Blockers on [Ca²⁺]ᵢ

It is increasingly appreciated that the procoagulant response depends on a persistent elevation of [Ca²⁺]ᵢ. Therefore, inhibition of the procoagulant response by Gardos channel inhibitors is expected to reduce the collagen-thrombin induced change in [Ca²⁺]ᵢ. To test this hypothesis we have conducted [Ca²⁺]ᵢ measurements in collagen plus thrombin activated platelets that were incubated with clotrimazol, charybdotoxin or quinine. In agreement with the notion that a sustained, threshold [Ca²⁺]ᵢ is of importance, [Ca²⁺]ᵢ levels, measured 5 minutes after stimulation with collagen plus thrombin either in the absence or presence of inhibitors, were compared. In the absence of inhibitors the change in [Ca²⁺]ᵢ was 364±18 nmol/L. When platelets were preincubated with clotrimazol (10 μmol/L), the increase in [Ca²⁺]ᵢ was significantly lower (Δ[Ca²⁺]ᵢ=275±10 nmol/L, P=0.01). This 24% reduction could be reversed with valinomycin (Δ[Ca²⁺]ᵢ=326±34 nmol/L). With charybdotoxin (20 nmol/L), no significant change in [Ca²⁺]ᵢ was found (Δ[Ca²⁺]ᵢ=360±23 nmol/L). The intrinsic fluorescence properties of quinine appeared to interfere with the Fura-2 signal, impeding determination of Δ[Ca²⁺]ᵢ with this inhibitor.

### Example 8

### The Impaired Procoagulant Response of Platelets from a Patient with Scott Syndrome can be Partially Corrected by Valinomycin

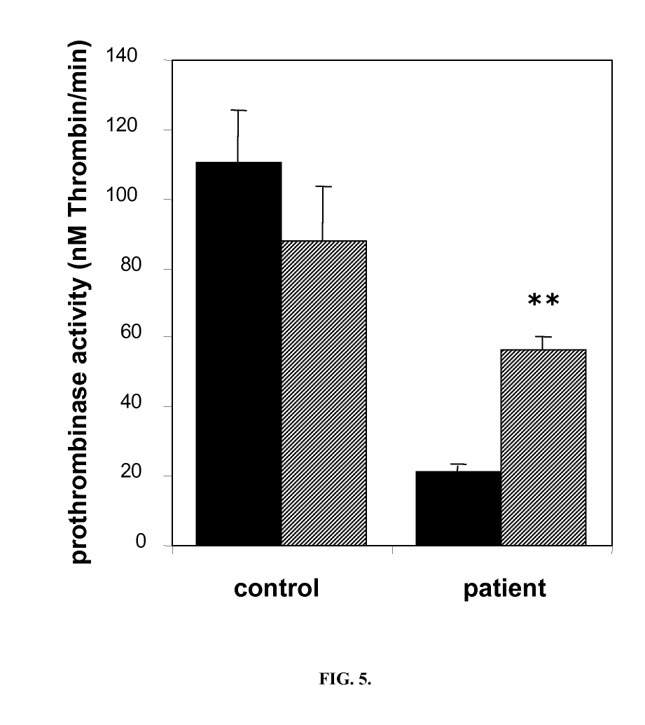

Platelets from patients with Scott syndrome elicit a partially impaired procoagulant response when activated with collagen plus thrombin and appear to be completely defective in their response to Ca²⁺-ionophore. Here, we used platelets from a Welsh Scott patient, whose clinical features have been described earlier.²⁶ Indeed, as shown in FIG. 5, the prothrombinase activity after stimulation with collagen and thrombin was approximately 20% of that of an identically treated ‘travel’ control.

In FIG. 5, the effect of valinomycin on collagen plus thrombin induced procoagulant activity of platelets from a patient with Scott syndrome is shown. Prothrombinase activities, expressed as nmol/L thrombin formed per minute, were determined after activation with collagen (10 μg/ml) plus thrombin (4 nmol/L) in the absence (black bars) and presence (hatched bars) of valinomycin (3 μmol/L). Data are mean values±1 S.D. (n=6).

Stimulation with collagen plus thrombin in the presence of 3 μmol/L valinomycin caused a significant increase in the procoagulant response of the Scott platelets, almost to the level of that observed for the control in the presence of valinomycin.

Thus, the impaired procoagulant response of platelets from a patient with Scott syndrome was partially restored by pretreatment with valinomycin, suggesting a possible defect of the Gardos channel in this syndrome. Promoting K⁺ efflux in platelets from Scott syndrome is thus a feasible strategy to address this bleeding disorder.

Interestingly, blood platelets from patients with this rare hereditary bleeding disorder characterized by a defective scramblase mechanism nevertheless expose PS upon activation in the presence of valinomycin. Notably, red cells from Scott syndrome, unlike normal red cells, have been shown to maintain their biconcave structure upon treatment with Ca²⁺-ionophore³⁵, whereas normal red cells maintain their biconcave structure with Ca²⁺-ionophore in the presence of Gardos-channel inhibitors.⁷ Therefore, it can be speculated that blood cells from Scott syndrome lack, or have defective Gardos channels, preventing operation of the scramblase mechanism, and thus the formation of a procoagulant platelet membrane surface.

The disclosure herein as a whole demonstrates that PS exposure, microvesiculation, and prothrombinase activity of platelets stimulated with collagen plus thrombin are markedly attenuated in the presence of specific inhibitors of the Gardos K⁺ channel, charybdotoxin, clotrimazol, and quinine, as well as in the presence of high extracellular K⁺. This inhibition does not occur with inhibitors of the non-Gardos K⁺ channels (e.g. apamin and iberiotoxin), suggesting that functional Gardos channels are a requisite for the process of PS externalization.

Collectively, these results provide evidence for the involvement of efflux of K⁺ ions through Ca²⁺-activated K⁺ channels in the procoagulant response of platelets, opening potential strategies for therapeutic interventions.

The invention now being generally described, it will be more readily understood by reference to the following examples which are included merely for purposes of illustration of certain aspects and embodiments of the present invention, and are not intended to limit the invention. All citations are expressly incorporated by reference herein.

### REFERENCES

1. Heemskerk J W, Bevers E M, Lindhout T. Platelet activation and blood coagulation. Thromb Haemost. 2002; 88:186-193.
2. Sims P J, Wiedmer T. Unraveling the mysteries of phospholipid scrambling. Thromb Haemost. 2001; 86:266-275.
3. Bevers E M, Comfurius P, Dekkers D W, Zwaal R F. Lipid translocation across the plasma membrane of mammalian cells. Biochim Biophys Acta. 1999; 1439:317-330.
4. Zhou Q, Sims P J, Wiedmer T. Expression of proteins controlling transbilayer movement of plasma membrane phospholipids in the B lymphocytes from a patient with Scott syndrome. Blood. 1998; 92:1707-1712.
5. Zhou Q, Zhao J, Wiedmer T, Sims P J. Normal hemostasis but defective hematopoietic response to growth factors in mice deficient in phospholipid scramblase 1. Blood. 2002; 99:4030-4038.
6. Keuren J F, Wielders S J, Ulrichts H, et al. Synergistic effect of thrombin on collagen-induced platelet procoagulant activity is mediated through protease-activated receptor-1. Arterioscler Thromb Vasc Biol. 2005; 25:1499-1505.
7. Lang P A, Kaiser S, Myssina S, Wieder T, Lang F, Huber S M. Role of Ca²⁺-activated K⁺ channels in human erythrocyte apoptosis. Am J Physiol Cell Physiol. 2003; 285:C1553-1560.
8. Zwaal R F, Comfurius P, Bevers E M. Scott syndrome, a bleeding disorder caused by defective scrambling of membrane phospholipids. Biochim Biophys Acta. 2004; 1636:119-128.
9. Rosing J, van Rijn J L, Bevers E M, van Dieijen G, Comfurius P, Zwaal R F. The role of activated human platelets in prothrombin and factor X activation. Blood. 1985; 65:319-332.
10. Heemskerk J W, Comfurius P, Feijge M A, Bevers E M. Measurement of the platelet procoagulant response. Methods Mol. Biol. 2004; 272:135-144.
11. Sims P J, Wiedmer T, Esmon C T, Weiss H J, Shattil S J. Assembly of the platelet prothrombinase complex is linked to vesiculation of the platelet plasma membrane. Studies in Scott syndrome: an isolated defect in platelet procoagulant activity. J Biol. Chem. 1989; 264:17049-17057.
12. Morel O, Toti F, Hugel B, Freyssinet J M. Cellular microparticles: a disseminated storage pool of bioactive vascular effectors. Curr Opin Hematol. 2004; 11:156-164.
13. Comfurius P, Senden J M, Tilly R H, Schroit A J, Bevers E M, Zwaal R F. Loss of membrane phospholipid asymmetry in platelets and red cells may be associated with calcium-induced shedding of plasma membrane and inhibition of aminophospholipid translocase. Biochim Biophys Acta. 1990; 1026:153-160.
14. Grynkiewicz G, Poenie M, Tsien R Y. A new generation of Ca²⁺ indicators with greatly improved fluorescence properties. J Biol. Chem. 1985; 260:3440-3450.
15. Di Cera E. Thrombin: a paradigm for enzymes allosterically activated by monovalent cations. C R Biol. 2004; 327:1065-1076.
16. Bucki R, Pastore J J, Giraud F, Janmey P A, Sulpice J C. Involvement of the Na⁺/H⁺ exchanger in membrane phosphatidylserine exposure during human platelet activation. Biochim Biophys Acta. Prepublished on Jan. 17, 2006. DOI 10.1016/j.bbalip.2005.12.008
17. Wolfs J L, Comfurius P, Rasmussen J T, et al. Activated scramblase and inhibited aminophospholipid translocase cause phosphatidylserine exposure in a distinct platelet fraction. Cell Mol Life Sci. 2005; 62:1514-1525.
18. Maher A D, Kuchel P W. The Gardos channel: a review of the Ca²⁺-activated K⁺ channel in human erythrocytes. Int J Biochem Cell Biol. 2003; 35:1182-1197.
19. Allan D, Thomas P. Ca²⁺-induced biochemical changes in human erythrocytes and their relation to microvesiculation. Biochem J. 1981; 198:433-440.
20. Armando-Hardy M, Ellory J C, Ferreira H G, Fleminger S, Lew V L. Inhibition of the calcium-induced increase in the potassium permeability of human red blood cells by quinine. J. Physiol. 1975; 250:32P-33P.
21. Reichstein E, Rothstein A. Effects of quinine on Ca⁺⁺-induced K⁺ efflux from human red blood cells. J Membr Biol. 1981; 59:57-63.
22. Dachary-Prigent J, Pasquet J M, Freyssinet J M, Nurden A T. Calcium involvement in aminophospholipid exposure and microparticle formation during platelet activation: a study using Ca²⁺-ATPase inhibitors. Biochemistry. 1995; 34:11625-11634.
23. London F S, Marcinkiewicz M, Walsh P N. A subpopulation of platelets responds to thrombin- or SFLLRN-stimulation with binding sites for factor IXa. J Biol. Chem. 2004; 279:19854-19859.
24. Augereau O, Rossignol R, DeGiorgi F, Mazat J P, Letellier T, Dachary-Prigent J. Apoptotic-like mitochondrial events associated to phosphatidylserine exposure in blood platelets induced by local anaesthetics. Thromb Haemost. 2004; 92:104-113.
25. Wada A, Sakurai S, Kobayashi H, Yanagihara N, Izumi F. Suppression by phospholipase A₂ inhibitors of secretion of catecholamines from isolated adrenal medullary cells by suppression of cellular calcium uptake. Biochem Pharmacol. 1983; 32:1175-1178.
26. Munnix I C, Harmsma M, Giddings J C, et al. Store-mediated calcium entry in the regulation of phosphatidylserine exposure in blood cells from Scott patients. Thromb Haemost. 2003; 89:687-695.
27. Fine B P, Hansen K A, Salcedo J R, Aviv A. Calcium-activated potassium channels in human platelets. Proc Soc Exp Biol Med. 1989; 192:109-113.
28. Pipili E. Platelet membrane potential: simultaneous measurement of diSC3(5) fluorescence and optical density. Thromb Haemost. 1985; 54:645-649.
29. Mahaut-Smith M P, Rink T J, Collins S C, Sage S O. Voltage-gated potassium channels and the control of membrane potential in human platelets. J Physiol. 1990; 428:723-735.
30. Mahaut-Smith M P. Calcium-activated potassium channels in human platelets. J Physiol. 1995; 484 (Pt 1):15-24.
31. McManus O B. Calcium-activated potassium channels: regulation by calcium. J Bioenerg Biomembr. 1991; 23:537-560.
32. Castle N E. Recent advances in the biology of small conductance calcium-activated potassium channels. Perspect Drug Discov. 1999; 15/16:131-154.
33. Rodriguez de la Vega R C, Possani L D. Current views on scorpion toxins specific for K⁺-channels. Toxicon. 2004; 43:865-875.
34. Gardos G. The function of calcium in the potassium permeability of human erythrocytes. Biochim Biophys Acta. 1958; 30:653-654.
35. Bevers E M, Wiedmer T, Comfurius P, et al. Defective Ca²⁺-induced microvesiculation and deficient expression of procoagulant activity in erythrocytes from a patient with a bleeding disorder: a study of the red blood cells of Scott syndrome. Blood. 1992; 79:380-388.
36. Williamson P, Christie A, Kohlin T, et al. Phospholipid scramblase activation pathways in lymphocytes. Biochemistry. 2001; 40:8065-8072.

What is claimed is:

1. A method of modulating platelet procoagulant response comprising:
exposing a platelet to an agent that modulates a Ca²⁺-sensitive K⁺ channel of the platelet.

2. The method of claim 1, wherein the agent does not interfere or interferes minimally with the bleeding arrest function of the platelet.

3. The method of claim 1, wherein the agent inhibits the channel.

4. The method of claim 1, wherein the agent is at least one compound selected from the group consisting of clotrimazol, charybdotoxin, quinine, or biologically active analogs thereof.

5. The method of claim 2, wherein the agent is at least one compound selected from the group consisting of clotrimazol, charybdotoxin, or biologically active analogs thereof.

6. The method of claim 3 further comprising reversing the inhibition of the channel.

7. The method of claim 6, wherein the step of reversing the inhibition is performed with valinomycin or a biologically active analog thereof.

8. A method of modulating platelet procoagulant response while maintaining platelet bleeding arrest function, the method comprising:
exposing a platelet to an elevated extracellular K⁺ concentration.

9. A method of treating a patient for a disorder involving platelet procoagulant response, the method comprising:
selecting a patient in need of treatment for a disorder involving platelet procoagulant response, and
administering to the patient a therapeutically effective amount of an agent that modulates Ca²⁺-sensitive K⁺ channels of platelets.

10. The method of claim 9, wherein the agent does not interfere or interferes minimally with the bleeding arrest capabilities of the patient.

11. The method of claim 9, wherein the disorder is selected from the group consisting of acute coronary syndromes, percutaneous intervention, cardiac bypass surgery (CABG), atrial fibrillation, deep vein thrombosis, intermittent claudication, peripheral arterial disease, atherosclerosis, and thrombocytopenia or other bleeding diatheses.

12. The method of claim 9, wherein the agent inhibits the channels.

13. The method of claim 9, wherein the agent is at least one compound selected from the group consisting of clotrimazol, charybdotoxin, quinine, or biologically active analogs thereof

14. The method of claim 10, wherein the agent is at least one compound selected from the group consisting of clotrimazol, charybdotoxin, or biologically active analogs thereof.

15. The method of claim 9, wherein the agent is administered in combination with a secondary therapeutic agent selected from the group consisting of aspirin, TP antagonist, thromboxane synthase inhibitor, P2Y12 antagonist, syk kinase inhibitor, and dipyramidole.

16. A method of identifying an agent useful for modulating platelet procoagulant response comprising the steps of:
exposing Ca²⁺-sensitive K⁺ channels of platelets to a candidate agent,
determining the modulatory effect of the candidate agent on the channels by comparing the K⁺ efflux through the channels in the presence of the candidate agent to the K⁺ efflux through the channels in the absence of the candidate agent, and
selecting the candidate agent as an agent useful for modulating platelet procoagulant response if the channels are modulated in the presence of the candidate agent at a level greater that than observed in the absence of the candidate agent.

17. A method of identifying an agent useful for modulating platelet procoagulant response, the method comprising:
exposing Ca²⁺-sensitive K⁺ channels of platelets to a candidate agent,
comparing the modulatory effect of the candidate agent on the channels with the modulatory effect of at least one reference compound on the channels, and
selecting the candidate agent as an agent useful for modulating platelet procoagulant response if its modulatory effect is at least as great as the modulatory effect of the reference compound.

18. The method of claim 17, wherein the reference compound is selected from clotrimazol, charybdotoxin, and quinine, and biologically active analogs thereof.

19. The method of claim 17, wherein the candidate agent does not interfere or interferes minimally with the bleeding arrest capabilities of platelets.

20. The method of claim 17, wherein the candidate agent has no or minimal effect on platelet aggregation or release of ATP.

21. The method of claim 17, wherein the agent inhibits the channels.

22. A method of treating a patient with Scott syndrome comprising exposing a patient with Scotts syndrome to an agent that promotes K⁺ efflux in platelets.

23. The method of claim 22, wherein the agent that promotes K⁺ efflux in platelets is a K⁺ ionophore.

24. The method of claim 23, wherein the K⁺ ionophore is valinomycin.

25. The use of an agent that modulates Ca²⁺-sensitive K⁺ channels in the manufacture a medicament for treatment of a patient for a disorder involving platelet procoagulant response.

26. The use of claim 25, wherein the agent is selected from among clotrimazol, charybdotoxin and quinine, and biologically active analogs thereof.
